(19) United States
(12) Patent Application Publication
(10) Pub. No.: US 2026/0355096 A1
(43) Pub. Date: Nov. 12, 2026

(54) HYBRID NAIL POLISHES AND METHODS FOR MAKING AND APPLYING THE SAME

(71) Applicant: Mycone Dental Supply Co., Inc. d/b/a Keystone Cosmetics & Sciences, Gibbstown, NJ (US)

(72) Inventor: Samantha Steffier, Moorestown, NJ (US)

(73) Assignee: Mycone Dental Supply Co., Inc. d/b/a Keystone Cosmetics & Sciences, Gibbstown, NJ (US)

(21) Appl. No.: 19/667,696

(22) Filed: May 5, 2026

Related U.S. Application Data
(60) Provisional application No. 63/800,499, filed on May 6, 2025.

(51) Int. Cl. A61K 8/81; A61K 8/19; A61K 8/34; A61K 8/36; A61K 8/37; A61K 8/41; A61K 8/46; A61K 8/55; A61K 8/73; A61K 8/895; A61Q 3/02

(52) U.S. Cl. CPC A61K 8/8147; A61K 8/19; A61K 8/347; A61K 8/36; A61K 8/37; A61K 8/416; A61K 8/463; A61K 8/55; A61K 8/731; A61K 8/895; A61Q 3/02; A61K 2800/43; A61K 2800/81; A61K 2800/884

(57) ABSTRACT

A hybrid nail polish for the cosmetic adornment of fingernails, toenails, artificial nails, and artificial nail extensions, along with the methods for making and applying the same. Like inexpensive nail lacquers, the hybrid nail polish is easy to use and able to air-dry quickly without using artificial ultraviolet light, but still provides the durability of more expensive gel polish, which requires more time and artificial ultraviolet light to fully cure.

### FIELD OF INVENTION

The present inventions relate to field of nail polish, useful for the cosmetic adornment of fingernails, toenails, artificial nails, and artificial nail extensions (collectively, “Nails”).

### BACKGROUND

Until recently, two basic types of nail polish have existed in the marketplace: (1) simple nail lacquers, which are relatively inexpensive, easy for the consumer to self-apply and self-remove, air-dry quickly, but lack long-term durability and generally require re-application after approximately seven days of wear to maintain optimal appearance; and (2) gel polish, which tends to be more expensive, traditionally required professional application and an artificial ultraviolet (“UV”) or Light-Emitting Diode (“LED”) light source to fully cure the polish (to prevent irritation to the nails and the surrounding tissues). Gel polish could be difficult and time-consuming to remove, but generally provided superior appearance and durability (usually lasting for weeks without chipping or requiring reapplication).

More recently, nail polish consumers have demanded a product that provides the benefits of both types of polish, while minimizing their respective shortcomings. Accordingly, manufacturers of nail polish have sought to create “hybrid” nail polishes—with limited success—that provide the durability and superior appearance of a gel polish, that are relatively inexpensive, easy for the consumer to self-apply and self-remove, air dry quickly, do not irritate nails or the surrounding tissues, and do not necessarily require an artificial UV/LED light source to fully cure (“Hybrid Nail Polish”). Indeed, the nail polish industry has produced an abundance of research, patents, and patent applications—which are hereby incorporated by reference as though set forth at length—that are relevant to addressing these unmet needs demanded by the nail polish marketplace: U.S. Pat. Nos. 9,713,585 B2; 8,367,742 B2; 8,367,045 B2; 8,263,677; 8,399,537; 8,541,482; 9,023,326; 8,901,199; 10,149,807; EP 4,371,618 A2; US 2017/0312793 A1; and US 2015/0190331 A1.

The present inventions solve this dilemma by providing a “Hybrid Nail Polish” that contains novel formulations comprised of the following components: (1) a “Solvent,” such as Butyl Acetate, Ethyl Acetate, Isopropyl Acetate, Acetone, Isopropyl Alcohol, or Ethyl Alcohol; (2) a “Film Former,” such as Cellulose Acetate Propionate, Cellulose Acetate Butyrate, Nitrocellulose or [polyester resins, copolymers, cellulose & cellulosics, polyvinyl butyral, vinyl acetate polymers and copolymers, vinyl alcohol polymers and copolymers, vinyl chloride polymers and copolymers, styrene copolymers (e.g., styrene-butadiene copolymers, styrene-ethylene-butylene-styrene block copolymers, styrene-ethylene-propylene-styrene block copolymers, and the like), polyester polymers and copolymers, aldehyde resins, alkyd resins, acrylic polymers and polyurethane polymers and copolymers]; (3) an optional “Inhibitor,” such as 2,6-di-tert-butyl-4-methylphenol [hydroquinone, 4-methoxyphenol]; (4) a “Monomer,” such as Ethoxylated Trimethylolpropane Triacrylate, or other Acrylate/Methacrylate monomers-mono, di, tri, tetra, pentafunctional; (5) a commercially available “Resin,” such as Polyether Urethane Methacrylate and Difunctional (Meth)Acrylate or Multifunctional Meth (Acrylate); (6) an “Initiator,” such as Ethyl (2,4,6-trimethylbenzoyl)phenylphosphinate (TPO-L), Phenylbis(2,4,6-trimethylbenzoyl) phosphineoxide (BAPO), 2-Hydroxy-2-methyl-1-phenyl-propan-1-one (1173), Methyl benzoylformate (MBF), Diphenyl(2,4,6-trimethylbenzoyl)phosphine oxide (TPO), (2,4,6-trimethylbenzoyl)-bis(4-methylphenyl)phosphinyl oxide (TMO), and 1-hydroxycyclohexyl phenyl ketone (HCPK, 184).’ (7) a “Thickener” (Stearalkonium Hectorite, Bentonite, Polyvinyl Butyral, Acrylates Copolymer, and Silica); (8) a “Conditioning Agents” (such as crotonic acid, vinyl C8-12 isoalkyl esters, VA, vitamins, biotin, oils and natural extracts, silk amino acids, proteins, and bis-vinyldimethicone crosspolymer) and (9) an optional “Colorant” (Pigments, Dyes, and effect additives)

### DETAILED DESCRIPTION

Certain terminology is used in the following description for convenience only and is not limiting. Similarly, this detailed description provides specific examples of preferred embodiments and does not limit the scope of the claimed inventions. Moreover, the descriptions of example embodiments do not exclude alternative embodiments of the inventions, as would be appreciated by one of ordinary skill in the art.

A person of ordinary skill in the art of nail aesthetics preferably will apply at least three separate coats of the Hybrid Nail Polish (“Coat,” or collectively, “Coats”) to the Nails to ensure superior appearance and durability: (1) a first Coat, the “Base Coat,” applied directly to the Nails to ensure sufficient adherence, durability, and a stable substrate for the application of subsequent coats of Hybrid Nail Polish; (2) a second Coat, the “Color Coat,” to enhance the appearance and depth of color and durability of the Nails; and (3) a third coat, the “Top Coat,” to enhance the appearance and gloss, and maximize durability of the Nails to prevent chipping, scratching or flaking of the applied Coats of Hybrid Nail Polish. Preferably, each Coat is separately formulated to optimize the characteristics of its intended purpose.

For increased strength and durability, the Base Coat optionally can be cured by exposure to natural light for approximately 1 minute to 10 minutes or with an artificial (UV or LED) light source for approximately 10 seconds to 10 minutes.

After the Base Coat is optionally cured, a Hybrid Nail Polish Color Coat is applied to the Base Coat to enhance the appearance and depth of color and durability of the Nails. For increased strength and durability, the Color Coat optionally can be cured by exposure to natural light for approximately 1 minute to 10 minutes or with an artificial (UV or LED) light source for approximately 10 seconds to 10 minutes. Multiple coats of color can be applied.

After the Color Coat is optionally cured, a Hybrid Nail Polish Top Coat is applied to the Color Coat to enhance appearance, durability, and gloss of the Nails and to prevent chipping and flaking. For increased strength and durability, the Top Coat optionally can be cured by exposure to natural light for approximately 1 minute to 10 minutes or with an artificial (UV or LED) light source for approximately 10 seconds to 10 minutes.

An alternative procedure for this system would involve the application of each product in sequence and optionally cured only once by exposure to natural light for approximately 1 minute to 10 minutes or with an artificial (UV or LED) light source for approximately 10 seconds to 10 minutes after all desired Coats have been applied.

It should be noted that each of the Hybrid Nail Polish Coats can be used independently from each other, depending on the specific need of user. For instance, a user may decide to apply only a Top Coat to the Nails in order to enhance durability and gloss. Each of the formulations (base, color, and top) can be used independently or in combination with one another and in combination with other commercial nail product that exist in the marketplace. For example, a user may apply a simple nail lacquer to the Nails and then apply a Top Coat to the Nails in order to enhance durability and gloss of the Nails.

### General Formulation of the Hybrid Nail Polish Base Coat

The Hybrid Nail Polish used to form a Base Coat is formulated to optimize adherence, durability, and to provide a stable substrate for the application of subsequent coats of Hybrid Nail Polish. It may be comprised of the following components (or alternatives) and in the following ranges of percentage by mass:

| | COMPONENT, ALTERNATIVES, AND COMBINATIONS | RANGE |
|---|---|---|
| CLASS | THEREOF | % MASS |
| Solvent | Ethyl Acetate; Butyl Acetate; Isopropyl Acetate; | 30-45% |
| | Acetone; Isopropyl Alcohol; and/or Ethyl Alcohol | |
| Inhibitor | 2,6-di-tert-butyl-4-methylphenol | 0-0.5% |
| Monomer | Ethoxylated trimethylolpropane tri(meth)acrylate; | 2.5-7.5% |
| | and/or Acrylate/Methacrylate monomers (mono, di, tri, | |
| | tetra, pentafunctional)] | |
| Initiator | Ethyl (2,4,6-trimethylbenzoyl) phenylphosphinate | 0.1-3% |
| | (TPO-L), Phenylbis(2,4,6-trimethylbenzoyl) | |
| | phosphineoxide (BAPO), 2-Hydroxy-2-methyl-1-phenyl- | |
| | propan-1-one (1173), Methyl benzoylformate (MBF), | |
| | Diphenyl(2,4,6-trimethylbenzoyl)phosphine oxide | |
| | (TPO), (2,4,6-trimethylbenzoyl)-bis(4- | |
| | methylphenyl)phosphinyl oxide (TMO), and 1- | |
| | hydroxycyclohexyl phenyl ketone (HCPK, 184) | |
| Resin | (meth)acrylate resin | 30-50% |
| Film Former | Nitrocellulose | 10-20% |
| Colorant | Pigments, Dyes, and Effect additives | 0-30% |

One specific example embodiment of a Hybrid Nail Polish used to form a Base Coat is formulated to optimize adherence, durability, and to provide a stable substrate for the application of subsequent coats of Hybrid Nail Polish. A preferred (but in no way limiting) embodiment of a Hybrid Nail Polish Base Coat formulation is comprised of the following components by percentage of mass:

### Example Hybrid Nail Polish Base Coat

| | | % |
|---|---|---|
| CLASS | COMPONENT | MASS |
| Solvent | Ethyl Acetate | 24.69 |
| Inhibitor | 2,6-di-tert-butyl-4-methylphenol | 0.09 |
| Monomer | Ethoxylated trimethylolpropane tri(meth)acrylate | 4.97 |
| Initiator | ethyl phenyl(2,4,6-trimethylbenzoyl)phosphinate | 1.98 |
| | and phenyl bis(2,4,6-trimethylbenzoyl)-phosphine | |
| | oxide | |
| Resin | (meth)acrylate resin | 34.52 |
| Film | Nitrocellulose Intermediate Solution | 33.76 |
| Former | | |

The following method was used for making this preferred embodiment of a Hybrid Nail Polish Base Coat: Ethyl Acetate is mixed with 2,6-di-tert-butyl-4-methylphenol using a high shear blade until the 2,6-di-tert-butyl-4-methylphenol is dissolved in the Ethyl Acetate to form a solution; next, a Nitrocellulose Intermediate and Polyether Urethane Methacrylate are mixed with the solution until a homogenous mixture is formed, using an overhead mixer; and, finally, Ethoxylated Trimethylolpropane Triacrylate and ethyl phenyl (2,4,6-trimethylbenzoyl) phosphinate and phenyl bis(2,4,6-trimethylbenzoyl)-phosphine oxide are added to the homogenous mixture and mixed for 3 to 5 minutes, using an overhead mixer.

It should be noted that a person of ordinary skill in the art will recognize that the Nitrocellulose Intermediate Solution can be pre-made by mixing NC Rs ¼ Sec. Nail Gr (NQ Prisma Cotton ¼ ES IPA 30%); with four parts Ethyl Acetate and one part N-Butyl-Acetate.

### General Formulation of the Hybrid Nail Polish Color Coat

The Hybrid Nail Polish used to form a Color Coat is formulated to enhance the appearance, depth of color, and durability of the Nails. It may be comprised of the following components (and/or alternatives) and in the following ranges of percentage by mass:

| | COMPONENT ALTERNATIVES, AND | |
|---|---|---|
| CLASS | COMBINATIONS THEREOF | % MASS |
| Solvent #1 | n-Butyl Acetate; Ethyl Acetate; Isopropyl Acetate; | 3-8% |
| | Acetone; Isopropyl Alcohol; and/or Ethyl Alcohol | |
| Solvent #2 | Ethyl Acetate; Butyl Acetate; Isopropyl Acetate; | 8-15% |
| | Acetone; Isopropyl Alcohol; and/or Ethyl Alcohol | |
| Inhibitor | 2,6-di-tert-butyl-4-methylphenol | 0-0.5% |
| Monomer | Ethoxylated trimethylolpropane tri(meth)acrylate; | 2.5-7.5% |
| | and/or Acrylate/Methacrylate monomers (mono, | |
| | di, tri, tetra, pentafunctional) | |
| Initiator | Ethyl (2,4,6-trimethylbenzoyl) phenylphosphinate | 0.1-5% |
| | (TPO-L), Phenylbis(2,4,6-trimethylbenzoyl) | |
| | phosphineoxide (BAPO), 2-Hydroxy-2-methyl-1- | |
| | phenyl-propan-1-one (1173), Methyl | |
| | benzoylformate (MBF), Diphenyl(2,4,6- | |
| | trimethylbenzoyl)phosphine oxide (TPO), (2,4,6- | |
| | trimethylbenzoyl)-bis(4-methylphenyl)phosphinyl | |
| | oxide (TMO), and 1-hydroxycyclohexyl phenyl | |
| | ketone (HCPK, 184) | |
| Resin | (meth)acrylate resin | 40-65% |
| Thickener | Stearalkonium Hectorite Intermediate Solution | 3-35% |
| Film Former | crotonic acid, vinyl C8-12 isoalkyl esters, Vinyl | 0.01-2.0% |
| | Acetate, and bis-vinyldimethicone crosspolymer | |
| Colorant | Pigments, dyes, and effect additives | 0-30% |

One example embodiment of a Hybrid Nail Polish used to form a Color Coat is formulated to enhance the appearance, depth of color, and durability of the Nails. A preferred (but in no way limiting) embodiment of a Hybrid Nail Polish Color Coat formulation is comprised of the following components by percentage of mass:

### Example Hybrid Nail Polish Color Coat

| | | % |
|---|---|---|
| CLASS | COMPONENT | MASS |
| Solvent #1 | n-Butyl Acetate | 4.61 |
| Solvent #2 | Ethyl Acetate | 11.03 |
| Inhibitor | 2,6-di-tert-butyl-4-methylphenol | 0.09 |
| Monomer | Ethoxylated trimethylolpropane tri(meth)acrylate | 3.72 |
| Initiator | ethyl phenyl(2,4,6-trimethylbenzoyl)phosphinate | 2.76 |
| | and phenyl bis(2,4,6-trimethylbenzoyl)-phosphine | |
| | oxide | |
| Resin | (meth)acrylate resin | 50.51 |
| Thickener | Stearalkonium Hectorite Intermediate Solution | 21.15 |
| Film | crotonic acid, vinyl C8-12 isoalkyl esters, Vinyl | 1.85 |
| Former | Acetate, and bis-vinyldimethicone crosspolymer | |
| Colorant | D&C Black #2 | 4.28 |

The following method was used for making this preferred embodiment of a Hybrid Nail Polish Color Coat: n-Butyl Acetate (Solvent #1) and Ethyl Acetate (Solvent #2) are mixed with 2,6-di-tert-butyl-4-methylphenol using a high shear blade until the 2,6-di-tert-butyl-4-methylphenol is dissolved to form a solution; next, Polyether Urethane Methacrylate and a Stearalkonium Hectorite intermediate are mixed with the solution until a first homogenous mixture is formed, using an overhead mixer; next, Ethoxylated Trimethylolpropane Triacrylate added to the first homogenous mixture and mixed for 3 to 5 minutes, using an overhead mixer until a second homogenous mixture is formed; and, finally, D&C Black #2, ethyl phenyl(2,4,6-trimethylbenzoyl)phosphinate and crotonic acid, vinyl C8-12 isoalkyl esters, Vinyl Acetate, and bis-vinyldimethicone crosspolymer are mixed with the second homogenous mixture until a third homogenous mixture is formed, using an overhead mixer.

It should be noted that a person of ordinary skill in the art will recognize that the Stearalkonium Hectorite Intermediate Solution can be pre-made by mixing NC Rs ¼ Sec. Nail Gr (NQ Prisma Cotton ¼ ES IPA 30%); with four parts Ethyl Acetate and one part N-Butyl-Acetate.

### General Formulation of the Hybrid Nail Polish Top Coat

The Hybrid Nail Polish used to form a Top Coat is formulated to enhance the appearance and gloss, and maximize durability of the Nails to prevent chipping, scratching or flaking. It may be comprised of the following components (and/or alternatives) and in the following ranges of percentage by mass:

| | COMPONENT ALTERNATIVES, AND | % |
|---|---|---|
| CLASS | COMBINATIONS THEREOF | MASS |
| Solvent | n-Butyl Acetate; Ethyl Acetate; Isopropyl | 0.5-5.0% |
| #1 | Acetate; Acetone; Isopropyl Alcohol; and/or | |
| | Ethyl Alcohol | |
| Solvent | Ethyl Acetate; Butyl Acetate; Isopropyl Acetate; | 40-65% |
| #2 | Acetone; Isopropyl Alcohol; and/or Ethyl Alcohol | |
| Film | Cellulose Acetate Propionate; and/or Cellulose | 5-20% |
| Former | Acetate Butyrate | |
| Inhibitor | 2,6-di-tert-butyl-4-methylphenol | 0-0.2% |
| Monomer | Ethoxylated trimethylolpropane tri(meth)acrylate; | 2.5-7.5% |
| | and/or Acrylate/Methacrylate monomers (mono, | |
| | di, tri, tetra, pentafunctional) | |
| Initiator | Ethyl (2,4,6-trimethylbenzoyl) phenylphosphinate | 0.1-5.0% |
| | (TPO-L), Phenylbis(2,4,6-trimethylbenzoyl) | |
| | phosphineoxide (BAPO), 2-Hydroxy-2-methyl-1- | |
| | phenyl-propan-1-one (1173), Methyl | |
| | benzoylformate (MBF), Diphenyl(2,4,6- | |
| | trimethylbenzoyl)phosphine oxide (TPO), (2,4,6- | |
| | trimethylbenzoyl)-bis(4-methylphenyl)phosphinyl | |
| | oxide (TMO), and 1-hydroxycyclohexyl phenyl | |
| | ketone (HCPK, 184) | |
| Resin | (meth)acrylate resin | 15-35% |
| Colorant | Pigments, Dyes, and Effect additives | 0-30% |

One example embodiment of a Hybrid Nail Polish used to form a Top Coat is formulated to enhance the appearance and gloss, and maximize durability of the Nails to prevent chipping, scratching or flaking of the applied coats Hybrid Nail Polish. A preferred (but in no way limiting) embodiment of a Hybrid Nail Polish Top Coat formulation is comprised of the following components by percentage of mass:

### Example Hybrid Nail Polish Top Coat

| | | % |
|---|---|---|
| CLASS | COMPONENT | MASS |
| Solvent | n-Butyl Acetate | 1.09 |
| Solvent | Ethyl Acetate | 62.79 |
| Film | Cellulose Acetate Propionate | 13.07 |
| Former | | |
| Monomer | Ethoxylated trimethylolpropane tri(meth)acrylate | 2.99 |
| Initiator | ethyl phenyl(2,4,6-trimethylbenzoyl)phosphinate | 2.07 |
| | and phenyl bis(2,4,6-trimethylbenzoyl)-phosphine | |
| | oxide | |
| Resin | (meth)acrylate resin | 17.98 |
| Colorant | D&C Violet #2 | 0.0001 |

The following method was used for making this preferred embodiment of a Hybrid Nail Polish Top Coat: n-Butyl Acetate (Solvent #1) and Ethyl Acetate (Solvent #2) are mixed with Cellulose Acetate Propionate using a high shear blade until the Cellulose Acetate Propionate is dissolved in the Solvents to form a solution; and, next, D&C Violet #2, Ethoxylated Trimethylolpropane Triacrylate, Polyether Urethane Methacrylate, and ethyl phenyl(2,4,6-trimethylbenzoyl)phosphinate and phenyl bis(2,4,6-trimethylbenzoyl)-phosphine oxide are mixed with the solution until a homogenous mixture is formed, using an overhead mixer.

What is claimed is:

1. A Hybrid Nail Polish Base Coat composition, comprising the following components with the following ranges of percentage of mass for the overall composition of:
one or more Solvents, collectively comprising 0.01-60% of the composition's mass;
optionally, one or more Inhibitors, comprising 0-1.0% of the composition's mass;
one or more Monomers, collectively comprising 0.01-40% of the composition's mass;
one or more Initiators, collectively comprising 0.01-10% of the composition's mass;
one or more Resins, collectively comprising 0.01-70% of the composition's mass;
one or more Film Formers, collectively comprising 0.01-50% of the composition's mass; and
optionally, one or more Colorants, collectively comprising 0-30% of the composition.

2. A Hybrid Nail Polish Base Coat composition, comprising the following components with the following ranges of percentage of mass for the overall composition of:
one or more Solvents, collectively comprising 30-45% of the composition's mass;
optionally, one or more Inhibitors, comprising 0-0.5% of the composition's mass;
one or more Monomers, collectively comprising 2.5-7.5% of the composition's mass;
one or more Initiators, collectively comprising 0.1-3% of the composition's mass;
one or more Resins, collectively comprising 30-50% of the composition's mass;
one or more Film Formers, collectively comprising 10-20% of the composition's mass; and
optionally, one or more Colorants, collectively comprising 0-5% of the composition.

3. A method of making the Hybrid Nail Polish Base Coat composition of claim 2, wherein:
one or more Solvents are mixed with one or more optional Inhibitors (if in composition) using a high shear blade until the Inhibitors are dissolved in the Solvents to form a solution;
one or more Film Formers and one or more Resins are mixed with the solution until a homogenous mixture is formed, using an overhead mixer; and
one or more Monomers, one or more optional Colorants, and one or more Initiators are added to the homogenous mixture and mixed for 3 to 5 minutes, using an overhead mixer.

4. A method of using the composition of claim 2, wherein the composition is applied directly to at least one Nail.

5. The method of using the composition of claim 2, wherein the composition is applied directly to at least one Nail and cured with naturally occurring light source to provide a Base Coat.

6. A method of using the composition of claim 2, wherein the composition is applied directly to at least one Nail and cured with an artificial light source to provide a Base Coat.

7. A Hybrid Nail Polish Color Coat composition, comprising the following components with the following ranges of percentage of mass for the overall composition:
one or more Solvents selected from the following solvents to form a first Solvent (Solvent #1), collectively comprising 3-8% of the composition's mass-n-Butyl Acetate; Ethyl Acetate; Isopropyl Acetate; Acetone; Isopropyl Alcohol; or Ethyl Alcohol;
one or more Solvents selected from the following solvents to form a second Solvent (Solvent #2), collectively comprising 8-15% of the composition's mass-Ethyl Acetate; Butyl Acetate; Isopropyl Acetate; Acetone; Isopropyl Alcohol; and/or Ethyl Alcohol;
one or more optional Inhibitors, comprising 0-0.5% of the composition's mass;
one or more Monomers, collectively comprising 2.5-7.5% of the composition's mass;
one or more Initiators, collectively comprising 0.5-5.0% of the composition's mass;
one or more Resins, collectively comprising 40-65% of the composition's mass;
one or more Thickeners, collectively comprising 0.01-35% of the composition's mass;
one or more Film Formers, collectively comprising 0.01-2% of the composition's mass; and
one or more optional Colorants, collectively comprising 0-30% of the composition's mass.

8. A method of making the Hybrid Nail Polish Color Coat composition of claim 7, wherein:
the first Solvent (Solvent #1) and the second Solvent (Solvent #2) are mixed with one or more optional Inhibitors (if in composition) using a high shear blade until the Inhibitors are dissolved in the Solvents to form a solution;
one or more Resins and one or more Thickeners are mixed with the solution until a first homogenous mixture is formed, using an overhead mixer; and
one or more Monomers are added to the first homogenous mixture and mixed for 3 to 5 minutes, using an overhead mixer until a second homogenous mixture is formed;
one or more Initiators, one of more optional Colorants, and one or more Film Formers are mixed with the second homogenous mixture until a third homogenous mixture is formed, using an overhead mixer.

9. A method of using the composition of claim 7, wherein the composition is applied to at least one Nail.

10. A method of using the composition of claim 7, wherein the composition is applied to at least one Nail and cured with naturally occurring light source to provide a Color Coat.

11. A method of using the composition of claim 7, wherein the composition is applied directly to at least one Nail and cured with an artificial light source to provide a Color Coat.

12. A Hybrid Nail Polish Top Coat composition, comprising the following components with the following ranges of percentage of mass for the overall composition:
one or more Solvents selected from the following solvents to form a first Solvent (Solvent #1), collectively comprising 0.5 to 2.0% of the composition's mass—n-Butyl Acetate; Ethyl Acetate; Isopropyl Acetate; Acetone; Isopropyl Alcohol; or Ethyl Alcohol;
one or more Solvents selected from the following solvents to form a second Solvent (Solvent #2), collectively comprising 50 to 65% of the composition's mass—Ethyl Acetate; Butyl Acetate; Isopropyl Acetate; Acetone; Isopropyl Alcohol; and/or Ethyl Alcohol;
one or more optional Inhibitors, comprising 0-1.0% of the composition's mass;
one or more Film Formers, collectively comprising 10 to 15% of the composition's mass.
one or more Monomers, collectively comprising 2.5 to 7.5% of the composition's mass;
one or more Initiators, collectively comprising 0.5 to 2.0% of the composition's mass; one or more Resins, collectively comprising 15 to 35% of the composition's mass; and
optionally, one or more Colorants, collectively comprising 0-5% of the composition.

13. A method of making the Hybrid Nail Polish Top Coat composition of claim 12, wherein:
the first Solvent (Solvent #1) and the second Solvent (Solvent #2) are mixed with one or more optional Inhibitors (if in composition) using a high shear blade until the Inhibitors are dissolved in the Solvents to form a solution;
one or more Film Formers using a high shear blade until the Film Formers are dissolved in the solution; and
one or more Monomers, one or more Resins, one or more optional Colorants, and one or more Initiators are mixed with the solution until a homogenous mixture is formed, using an overhead mixer.

14. A method of using the composition of claim 12, wherein the composition is applied to at least one Nail.

15. A method of using the composition of claim 12, wherein the composition is applied to at least one Nail and cured with naturally occurring light source to provide a Color Coat.

16. A method of using the composition of claim 12, wherein the composition is applied directly to at least one Nail and cured with an artificial light source to provide a Color Coat.

17. A Hybrid Nail Polish system wherein:
A Hybrid Nail Polish Base Coat composition (i.e., claim 1) is applied directly to at least one Nail and optionally cured to form a Base Coat;
a Hybrid Nail Polish Color Coat composition (i.e., claim 7) is applied to the Base Coat and optionally cured to form a Color Coat; and
a Hybrid Nail Polish Top Coat composition (i.e., claim 12) is applied to the Color Coat and optionally cured to form a Top Coat.

18. A Hybrid Nail Polish system wherein:
a Hybrid Nail Polish Base Coat composition (i.e., claim 1) is applied directly to at least one Nail and optionally cured with either a natural or artificial light source to form a Base Coat;
a Hybrid Nail Polish Color Coat composition (i.e., claim 7) is applied to the Base Coat and optionally cured with either a natural or artificial light source to form a Color Coat; and
a Hybrid Nail Polish Top Coat composition (i.e., claim 12) is applied to the Color Coat and optionally cured with either a natural or artificial light source to form a Top Coat.

19. A Hybrid Nail Polish system wherein:
a Hybrid Nail Polish Base Coat composition (i.e., claim 1) is applied directly to at least one Nail to form a Base Coat;
a Hybrid Nail Polish Color Coat composition (i.e., claim 7) is applied to the Base Coat to form a Color Coat; and
a Hybrid Nail Polish Top Coat composition (i.e., claim 12) is applied to the Color Coat to form a Top Coat; and optionally cured with either a natural or artificial light source to complete the application.

20. A Hybrid Nail Polish system wherein:
the system exceeds the performance of traditional nail lacquers, exhibiting perfect wear for 10 days.

21. A Hybrid Nail Polish system wherein:
the system exceeds the performance of UV curable nail gels on removal, removing in less than 5 minutes in a solvent comprised of one or more of the following: acetone, ethyl acetate, propyl acetate, butyl acetate, methyl ethyl ketone, propylene carbonate, mineral oil, vitamin oils, ethanol, and isopropyl alcohol.

22. The Hybrid Nail Polish system of claim 21 wherein the solvent is comprised of 0.1% to 60% acetone.

23. The Hybrid Nail Polish system of claim 21 wherein the solvent is comprised of 35% to 100% acetone.

24. A Hybrid Nail Polish system wherein:
the system requires light protectant and/or coated packaging to ensure the stability and performance of the material.

25. A Hybrid Nail Polish Kit, wherein a quantity of a Hybrid Nail Polish Base Coat composition (i.e., claim 1), a quantity of the Hybrid Nail Polish Color Coat composition (i.e., claim 7), and a quantity of the Hybrid Nail Polish Color Top composition (i.e., claim 12) are packaged or sold together, optionally along with a polish applicator or a selection of polish applicators, to use the System of claim 17.
